(19) United States
(12) Patent Application Publication
(10) Pub. No.: US 2008/0213749 A1
(43) Pub. Date: Sep. 4, 2008

(54) Compositions and methods for detecting human metapneumovirus

(76) Inventors: Melanie Feola, Cherry Hill, NJ (US); Larisa Gofman, Toms River, NJ (US); Eli Mordechai, Robbinsville, NJ (US); Martin E. Adelson, Hillsborough, NJ (US)

Correspondence Address: MEDICAL DIAGNOSTIC LABORATORIES LLC, 2439 KUSER ROAD, HAMILTON, NJ, 08690-3303, US

(21) Appl. No.: 11/713,408

(22) Filed: Mar. 2, 2007

(51) Int. Cl. C07H 21/00; C12Q 1/68; C12Q 1/70

(52) U.S. Cl. 435/5; 435/6; 536/22.1

(57) ABSTRACT

Disclosed are oligonucleotides useful in methods for determining whether a sample contains a human metapneumovirus or has an increased likelihood of containing a human metapneumovirus, a virus which is a causative agent of respiratory tract disease in humans. These oligonucleotides, which have nucleotide sequences derived from a coding segment of the gene encoding the fusion protein of a human metapneumovirus, are useful as forward and reverse primers for a polymerase chain reaction using reverse transcripts of RNA from a biological sample as templates, and as probes for detecting any resultant amplicon. Detection of an amplicon indicates the sample contains a human metapneumovirus or has an increased likelihood of containing a human metapneumovirus.

### BACKGROUND OF THE INVENTION

The following two publications describe methods for detecting human metapneumoviruses: (1) Maertzdorf et al., 2004, Real-time reverse transcriptase PCR assay for detection of human metapneumoviruses from all known genetic lineages. Journal of Clinical Microbiology 42(3):981-986, and (2) United States Patent Application Number 2006/0014140 entitled “Molecular Methods and Compositions for Detecting and Quantifying Respiratory Viruses.” The entire contents of these two publications are hereby incorporated by reference.

### SUMMARY OF THE INVENTION

Human metapneumovirus is a causative agent of respiratory tract disease in humans. Thus, there is a need for compositions and methods, e.g., oligonucleotides useful as primers and probes in polymerase chain reactions (“PCRs”), for detecting human metapneumovirus in a patient.

### OLIGONUCLEOTIDES OF THE INVENTION

An embodiment of the invention is drawn to an isolated oligonucleotide (e.g., a forward primer, a reverse primer, or a probe such as a molecular beacon) capable of hybridizing under highly stringent hybridization conditions to at least part of a segment of a polynucleotide, wherein the segment consists of nucleotides 661 through 683 of SEQ ID NO:2, SEQ ID NO:4, SEQ ID NO:6, or SEQ ID NO:8; nucleotides 623 through 651 of SEQ ID NO:2, SEQ ID NO:4, SEQ ID NO:6, or SEQ ID NO:8, nucleotides 1078 through 1098 of SEQ ID NO:1, SEQ ID NO:3, SEQ ID NO:5, or SEQ ID NO:7, or nucleotides 970 through 998 of SEQ ID NO:1, SEQ ID NO:3, SEQ ID NO:5, or SEQ ID NO:7.

The nucleotide sequence of SEQ ID NO:1 encodes the fusion protein of human metapneumovirus isolate 00-1 (van den Hoogen et al., 2001, A newly discovered human pneumovirus isolated from young children with respiratory tract disease. Nature Medicine 7:719-724; National Center for Biotechnology Information accession no. 20150834); the nucleotide sequence of SEQ ID NO:2 is the reverse complement of the nucleotide sequence of SEQ ID NO:1. The nucleotide sequence of SEQ ID NO:3 encodes the fusion protein of human metapneumovirus isolate CAN97-83 (Pham et al., 2005, Chimeric recombinant human metapneumoviruses with the nucleoprotein or phosphoprotein open reading frame replaced by that of avian metapneumovirus exhibit improved growth in vitro and attenuation in vivo. Journal of Virology 79:15114-15122; National Center for Biotechnology Information accession no. 34420896); the nucleotide sequence of SEQ ID NO:4 is the reverse complement of the nucleotide sequence of SEQ ID NO:3. The nucleotide sequence of SEQ ID NO:5 encodes the fusion protein of human metapneumovirus isolate NL/1/99 (Herfst et al., 2004, Recovery of human metapneumovirus genetic lineages A and B from cloned cDNA. Journal of Virology 78:8264-8270; National Center for Biotechnology Information accession no. 50059145); the nucleotide sequence of SEQ ID NO:6 is the reverse complement of the nucleotide sequence of SEQ ID NO:5. The nucleotide sequence of SEQ ID NO:7 encodes the fusion protein of human metapneumovirus isolate CAN98-75 (Biacchesi et al., 2003, Genetic diversity between human metapneumovirus subgroups. Virology 315:1-9; National Center for Biotechnology Information accession no. 34420886); the nucleotide sequence of SEQ ID NO:8 is the reverse complement of the nucleotide sequence of SEQ ID NO:7.

Highly stringent hybridization conditions include the following conditions: 6×SSC and 65° C.; highly stringent hybridization conditions described in Ausubel et al., 2002, Short Protocols in Molecular Biology, 5^{th} edition, Volumes 1 and 2, John Wiley & Sons, Inc., Hoboken, N.J., the entire contents of which are hereby incorporated by reference; and highly stringent hybridization conditions described in Ausubel et al., 1997, Short Protocols in Molecular Biology, 3^{rd} edition, John Wiley & Sons, Inc., New York, N.Y., the entire contents of which are hereby incorporated by reference.

In another embodiment, the oligonucleotide is at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical to, or is 100% identical to the reverse complement of a segment of a polynucleotide, wherein the segment consists of nucleotides 661 through 683 of SEQ ID) NO:2, SEQ ID NO:4, SEQ ID NO:6, or SEQ ID NO:8, nucleotides 623 through 651 of SEQ ID NO:2, SEQ ID NO:4, SEQ ID NO:6, or SEQ ID NO:8, nucleotides 1078 through 1098 of SEQ ID NO:1, SEQ ID NO:3, SEQ ID NO:5, or SEQ ID NO:7, or nucleotides 970 through 998 of SEQ ID NO:1, SEQ ID NO:3, SEQ ID NO:5, or SEQ ID NO:7.

Pairwise nucleotide sequence alignments and determination of percent identities are performed using the default parameters of the Clustal V algorithm or the Clustal W algorithm, wherein both algorithms are incorporated into the Power Macintosh MegAlign 6.1 program (DNASTAR, Madison, Wis.). The default parameters for pairwise alignments using the Clustal V algorithm are as follows: Ktuple=1, gap penalty=3, window=5, and diagonals=5. The default parameters for pairwise alignments using the Clustal W algorithm are as follows: gap penalty=10.00 and gap length=0.10. The Clustal V algorithm is described in Higgins et al., 1989, Fast and sensitive multiple sequence alignments on a microcomputer. Computer Applications in the Biosciences 5:151-153, the entire contents of which are hereby incorporated by reference. The Clustal W algorithm is described in Thompson et al., 1994, CLUSTAL W: improving the sensitivity of progressive multiple sequence alignment through sequence weighting, position specific gap penalties and weight matrix choice. Nucleic Acids Research 22:4673-80, the entire contents of which are hereby incorporated by reference. In another embodiment, the oligonucleotide and the segment of the polynucleotide contain the same number of nucleotides.

In another embodiment, the oligonucleotide comprises 8, 9, 10, 11, 12, 13, 14, 15, 16, 17, 18, 19, 20, 21, 22, 23, 24, 25, 26, 27, 28, 29, 30, 31, 32, 33, 34, 35, 36, 37,38, 39, 40, 41, 42, 43, 44, 45, 46, 47, 48, 49, or 50 nucleotides. In another embodiment, the oligonucleotide is from 8 to 50 nucleotides long, from 12 to 24 nucleotides long, from 15 to 50 nucleotides long, or from 25 to 35 nucleotides long. In another embodiment, the oligonucleotide comprises the nucleotide sequence of SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, or SEQ ID NO:18. In another embodiment, the oligonucleotide consists of the nucleotide sequence of SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, or SEQ ID NO:18.

### OLIGONUCLEATIDE COMBINATIONS OF THE INVENTION

Another embodiment of the invention is directed to a composition (e.g., a reaction mixture or a kit) containing a first isolated oligonucleotide (e.g., a forward primer) and a second isolated oligonucleotide (e.g., a reverse primer). In another embodiment, the first oligonucleotide is capable of hybridizing under highly stringent hybridization conditions to a polynucleotide consisting of the nucleotide sequence of SEQ ID NO:2, SEQ ID NO:4, SEQ ID NO:6, or SEQ ID NO:8. In another embodiment, the second oligonucleotide is capable of hybridizing under highly stringent hybridization conditions to a polynucleotide consisting of the nucleotide sequence of SEQ ID NO:1, SEQ ID NO:3, SEQ ID NO:5, or SEQ ID NO:7. In another embodiment, the first oligonucleotide is capable of hybridizing under highly stringent hybridization conditions to at least a part of a segment of a polynucleotide, wherein the segment consists of nucleotides 661 through 683 of SEQ ID NO:2, SEQ ID NO:4, SEQ ID NO:6, or SEQ ID NO:8. In another embodiment, the second oligonucleotide is capable of hybridizing under highly stringent hybridization conditions to at least part of a segment of a polynucleotide, wherein the segment consists of nucleotides 1078 through 1098 of SEQ ID NO:1, SEQ ID NO:3, SEQ ID NO:5, or SEQ ID NO:7.

In another embodiment, the first oligonucleotide is at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical to, or is 100% identical to a segment of a first polynucleotide based on the Clustal V or W alignment method using the default parameters, wherein the first polynucleotide consists of the nucleotide sequence of SEQ ID NO:1, SEQ ID NO:3, SEQ ID NO:5, or SEQ ID NO:7. In another embodiment, the first oligonucleotide and the segment of the first polynucleotide contain the same number of nucleotides. In another embodiment, the second oligonucleotide is at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical to, or is 100% identical to a segment of a second polynucleotide based on the Clustal V or W alignment method using the default parameters, wherein the second polynucleotide consists of the nucleotide sequence of SEQ ID NO:2, SEQ ID NO:4, SEQ ID NO:6, or SEQ ID NO:8. In another embodiment, the second oligonucleotide and the segment of the second polynucleotide contain the same number of nucleotides.

In another embodiment, the first oligonucleotide is at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical to, or is 100% identical to a segment of a polynucleotide, wherein the segment consists of the reverse complement of nucleotides 661 through 683 of SEQ ID NO:2, SEQ ID NO:4, SEQ ID NO:6, or SEQ ID NO:8 based on the Clustal V or W alignment method using the default parameters. In another embodiment, the second oligonucleotide is at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical to, or is 100% identical to a segment of a polynucleotide, wherein the segment consists of the reverse complement of nucleotides 1078 through 1098 of SEQ ID NO:1, SEQ ID NO:3, SEQ ID NO:5, or SEQ ID NO:7 based on the Clustal V or W alignment method using the default parameters.

In another embodiment, the first oligonucleotide consists of a nucleotide sequence comprised by the nucleotide sequence of SEQ ID NO:1, SEQ ID NO:3, SEQ ID NO:5, or SEQ ID NO:7. In another embodiment the second oligonucleotide consists of a nucleotide sequence comprised by the nucleotide sequence of SEQ ID NO:2, SEQ ID NO:4, SEQ ID NO:6, or SEQ ID NO:8. In another embodiment, the first or second oligonucleotide comprises 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides. In another embodiment, the first or second oligonucleotide is from 8 to 50 nucleotides long or from 12 to 24 nucleotides long. In another embodiment, the first oligonucleotide comprises the nucleotide sequence of SEQ ID NO:9, SEQ ID NO:10, SEQ ID NO:11, or SEQ ID NO:12. In another embodiment, the first oligonucleotide consists of the nucleotide sequence of SEQ,ID NO:9, SEQ ID NO:10, SEQ ID NO:11, or SEQ ID NO:12. In another embodiment, the second oligonucleotide comprises the nucleotide sequence of SEQ ID NO:13, SEQ ID NO:14, or SEQ ID NO:15. In another embodiment, the second oligonucleotide consists of the nucleotide sequence of SEQ ID NO:13, SEQ ID NO:14, or SEQ ID NO:15.

In another embodiment, the composition contains a third isolated oligonucleotide (e.g., a probe such as a molecular beacon). In another embodiment, the third oligonucleotide is capable of hybridizing under highly stringent hybridization conditions to a polynucleotide consisting of the nucleotide sequence of SEQ ID NO:1, SEQ ID NO:2, SEQ ID NO:3, SEQ ID NO:4, SEQ ID NO:5, SEQ ID NO:6, SEQ ID NO:7, or SEQ ID NO:8. In another embodiment, the third oligonucleotide is capable of hybridizing under highly stringent hybridization conditions to at least part of a segment of a polynucleotide, wherein the segment consists of nucleotides 623 through 651 of SEQ ID NO:2, SEQ ID NO:4, SEQ ID NO:6, or SEQ ID NO:8 or nucleotides 970 through 998 of SEQ ID NO:1, SEQ ID NO:3, SEQ ID NO:5, or SEQ ID NO:7.

In another embodiment, the third oligonucleotide is at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical to, or is 100% identical to a segment of a polynucleotide based on the Clustal V or W alignment method using the default parameters, wherein the polynucleotide consists of the nucleotide sequence of SEQ ID NO:1, SEQ ID NO:2, SEQ ID NO:3, SEQ ID NO:4, SEQ ID NO:5, SEQ ID NO:6, SEQ ID NO:7, or SEQ ID NO:8. In another embodiment, the third oligonucleotide and the segment of the polynucleotide contain the same number of nucleotides.

In another embodiment, the third oligonucleotide is at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical to, or is 100% identical to a segment of a polynucleotide, wherein the segment consists of the reverse complement of nucleotides 623 through 651 of SEQ ID NO:2, SEQ ID NO:4, SEQ ID NO:6, or SEQ ID NO:8 or nucleotides 970 through 998 of SEQ ID NO:1, SEQ ID NO:3, SEQ ID NO:5, or SEQ ID NO:7 based on the Clustal V or W alignment method using the default parameters.

In another embodiment, the third oligonucleotide consists of a nucleotide sequence comprised by the nucleotide sequence of SEQ ID NO:1, SEQ ID NO:2, SEQ ID NO:3, SEQ ID NO:4, SEQ ID NO:5, SEQ ID NO:6, SEQ ID NO:7, or SEQ ID NO:8. In another embodiment, the third oligonucleotide comprises 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides. In another embodiment, the third oligonucleotide is from 15 to 50 nucleotides long or from 25 to 35 nucleotides long. In another embodiment, the third oligonucleotide comprises the nucleotide sequence of SEQ ID NO:16, SEQ ID NO:17, or SEQ ID NO:18. In another embodiment, the third oligonucleotide consists of the nucleotide sequence of SEQ ID NO:16, SEQ ID NO:17, or SEQ ID NO:18. In another embodiment, a 6-carboxy-fluorescein moiety is attached to the 5′ end of the third oligonucleotide. In another embodiment, a Black Hole Quencher 1 moiety is attached to the 3′ end of the third oligonucleotide.

### METHODS OF THE INVENTION

Another embodiment of the invention concerns a method for determining whether a sample (e.g., a biological sample such as a nasal pharyngeal aspirate) contains a human metapneumovirus or has an increased likelihood of containing a human metapneumovirus, wherein the method comprises the following: (a) providing a vessel containing a composition, wherein the composition contains first and second primers, and a reverse transcript of an RNA from the sample, wherein the composition is capable of amplifying, by a polymerase chain reaction, a segment of the reverse transcript to produce an amplicon, wherein production of the amplicon is primed by the first and second primers, wherein the first primer is capable of hybridizing under highly stringent hybridization conditions to a polynucleotide consisting of the nucleotide sequence of SEQ ID NO:2, SEQ ID NO:4, SEQ ID NO:6, or SEQ ID NO:8, wherein the second primer is capable of hybridizing under highly stringent hybridization conditions to a polynucleotide consisting of the nucleotide sequence of SEQ ID NO:1, SEQ ID NO:3, SEQ ID NO:5, or SEQ ID NO:7; (b) incubating the vessel under conditions allowing production of the amplicon if the sample contains a human metapneumovirus, and (c) determining that the sample contains a human metapneumovirus if the amplicon is detected or that the sample has an increased likelihood of containing a human metapneumovirus if the amplicon is detected, or determining that the sample does not contain a human metapneumovirus if the amplicon is not detected or that the sample does not have an increased likelihood of containing a human metapneumovirus if the amplicon is not detected.

In another embodiment, the first primer is capable of hybridizing under highly stringent hybridization conditions to at least part of a segment of a polynucleotide, wherein the segment consists of nucleotides 661 through 683 of SEQ ID NO:2, SEQ ID NO:4, SEQ ID NO:6, or SEQ ID NO:8. In another embodiment, the second primer is capable of hybridizing under highly stringent hybridization conditions to at least part of a segment of a polynucleotide, wherein the segment consists of nucleotides 1078 through 1098 of SEQ ID NO:1, SEQ ID NO:3, SEQ ID NO:5, or SEQ ID NO:7.

In another embodiment, the first primer is at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical to, or is 100% identical to a segment of a first polynucleotide based on the Clustal V or W alignment method using the default parameters, wherein the first polynucleotide consists of the nucleotide sequence of SEQ ID NO:1, SEQ ID NO:3, SEQ ID NO:5, or SEQ ID NO:7. In another embodiment, the first primer and the segment of the first polynucleotide contain the same number of nucleotides. In another embodiment, the second primer is at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical to, or is 100% identical to a segment of a second polynucleotide based on the Clustal V or W alignment method using the default parameters, wherein the second polynucleotide consists of the nucleotide sequence of SEQ ID NO:2, SEQ ID NO:4, SEQ ID NO:6, or SEQ ID NO:8. In another embodiment, the second primer and the segment of the second polynucleotide contain the same number of nucleotides.

In another embodiment, the first primer is at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical to, or is 100% identical to a segment of a polynucleotide, wherein the segment consists of the reverse complement of nucleotides 661 through 683 of SEQ ID NO:2, SEQ ID NO:4, SEQ ID NO:6, or SEQ ID NO:8 based on the Clustal V or W alignment method using the default parameters. In another embodiment, the second primer is at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical to, or is 100% identical to a segment of a polynucleotide, wherein the segment consists of the reverse complement of nucleotides 1078 through 1098 of SEQ ID NO:1, SEQ ID NO:3, SEQ ID NO:5, or SEQ ID NO:7 based on the Clustal V or W alignment method using the default parameters.

In another embodiment, the first primer consists of a nucleotide sequence comprised by the nucleotide sequence of SEQ ID NO:1, SEQ ID NO:3, SEQ ID NO:5, or SEQ ID NO:7. In another embodiment the second primer consists of a nucleotide sequence comprised by the nucleotide sequence of SEQ ID NO:2, SEQ ID NO:4, SEQ ID NO:6, or SEQ ID NO:8. In another embodiment, the first or second primer comprises 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides. In another embodiment, the first or second primer is from 8 to 50 nucleotides long or from 12 to 24 nucleotides long. In another embodiment, the first primer comprises the nucleotide sequence of SEQ ID NO:9, SEQ ID NO:10, SEQ ID NO:11, or SEQ ID NO:12. In another embodiment, the first primer consists of the nucleotide sequence of SEQ ID NO:9, SEQ ID NO:10, SEQ ID NO:11, or SEQ ID NO:12. In another embodiment, the second primer comprises the nucleotide sequence of SEQ ID NO:13, SEQ ID NO:14, or SEQ ID NO:15. In another embodiment, the second primer consists of the nucleotide sequence of SEQ ID NO:13, SEQ ID NO:14, or SEQ ID NO:15.

Optionally, in (b) of the method, the vessel contains an oligonucleotide probe (e.g., a molecular beacon) capable of detecting the amplicon if the amplicon is produced in (b). In another embodiment, the oligonucleotide probe is capable of hybridizing under highly stringent hybridization conditions to a polynucleotide consisting of the nucleotide sequence of SEQ ID NO:1, SEQ ID NO:2, SEQ ID NO:3, SEQ ID NO:4, SEQ ID NO:5, SEQ ID NO:6, SEQ ID NO:7, or SEQ ID NO:8. In another embodiment, the oligonucleotide probe is capable of hybridizing under highly stringent hybridization conditions to at least part of a segment of a polynucleotide, wherein the segment consists of nucleotides 623 through 651 of SEQ ID NO:2, SEQ ID NO:4, SEQ ID NO:6, or SEQ ID NO:8 or nucleotides 970 through 998 of SEQ ID NO:1, SEQ ID NO:3, SEQ ID NO:5, or SEQ ID NO:7.

In another embodiment, the oligonucleotide probe is at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical to, or is 100% identical to a segment of a polynucleotide based on the Clustal V or W alignment method using the default parameters, wherein the polynucleotide consists of the nucleotide sequence of SEQ ID NO:1, SEQ ID NO:2, SEQ ID NO:3, SEQ ID NO:4, SEQ ID NO:5, SEQ ID NO:6, SEQ ID NO:7, or SEQ ID NO:8. In another embodiment, the oligonucleotide probe and the segment of the polynucleotide contain the same number of nucleotides.

In another embodiment, the oligonucleotide probe is at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical to, or is 100% identical to a segment of a polynucleotide, wherein the segment consists of the reverse complement of nucleotides 623 through 651 of SEQ ID NO:2, SEQ ID NO:4, SEQ ID NO:6, or SEQ ID NO:8 or nucleotides 970 through 998 of SEQ ID NO:1, SEQ ID NO:3, SEQ ID NO:5, or SEQ ID NO:7 based on the Clustal V or W alignment method using the default parameters. in another embodiment, the oligonucleotide probe consists of a nucleotide sequence comprised by the nucleotide sequence of SEQ ID NO:1, SEQ ID NO:2, SEQ ID NO:3, SEQ ID NO:4, SEQ ID NO:5, SEQ ID NO:6, SEQ ID NO:7, or SEQ ID NO:8. In another embodiment, the oligonucleotide probe comprises 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides. In another embodiment, the oligonucleotide probe is from 15 to 50 nucleotides long or from 25 to 35 nucleotides long. In another embodiment, the oligonucleotide probe comprises the nucleotide sequence of SEQ ID NO:16, SEQ ID NO:17, or SEQ ID NO:18. In another embodiment, the oligonucleotide probe consists of the nucleotide sequence of SEQ ID NO:16, SEQ ID NO:17, or SEQ ID NO:18. In another embodiment, a 6-carboxy-fluorescein moiety is attached to the 5′ end of the oligonucleotide probe. In another embodiment, a Black Hole Quencher 1 moiety is attached to the 3′ end of the oligonucleotide probe. In another embodiment, the amplicon is detected by the oligonucleotide probe during real-time PCR. In another embodiment, the amplicon is detected by gel electrophoresis after the PCR is completed.

### DETAILED DESCRIPTION

The following examples illustrate the use of the methods and compositions of the invention. These examples are set forth by way of illustration only, and nothing therein shall be taken as a limitation upon the overall scope of the invention.

### EXAMPLE 1

### PCR Conditions for Clinical Samples

For the testing of each clinical sample, the PCR was attempted in a volume of 25 ll containing the following: RNA extracted from the clinical sample (e.g., 2.0 ng), 50 U of iScript Reverse Transcriptase (Bio-Rad Laboratories, Inc., Hercules, Calif.), 600 nM of a first primer consisting of the nucleotide sequence of SEQ ID NO:12 (“Primer MPV6-Forward”); 600 nM of a second primer consisting of the nucleotide sequence of SEQ ID NO:15 (“Primer MPV6-Reverse”); 1200 nM of an oligonucleotide probe consisting of the nucleotide sequence of SEQ ID NO:18, wherein a 6-carboxy-fluorescein moiety and a Black Hole Quencher 1 moiety were attached to the 5′ end and 3′ end, respectively, of the oligonucleotide probe (“Probe MPV6-1”); and 1× RT-PCR Mastermix (Bio-Rad Laboratories, Inc., Hercules, Calif.), wherein a 2× stock solution of the RT-PCR Mastermix contained 5 U/μl of iTaq DNA polymerase, 400 nM MgCl₂, 400 nM dATP, 400 nM dCTP, 400 nM dGTP, 400 nM dTTP, and proprietary Bio-Rad Laboratories stabilizers. Probe MPV6-1 was present in the reaction mixture to monitor real-time synthesis of the amplicon resulting from each successful PCR. Primer MPV6-Forward, Primer MPV6-Reverse, and Probe MPV6-1 were synthesized by Integrated DNA Technologies (Stokie, Ill.). As indicated in the sequence listing, each of MPV6-Forward Primer, MPV6-Reverse Primer, and MPV6-Probe 1 was degenerate, i.e., was a genus of oligonucleotides, wherein all of the species in the genus were present at approximately the same concentration.

Nucleotide sequences of additional first primers, second primers, and oligonucleotide probes are determined from the nucleotide sequences of SEQ ID NO:1, SEQ ID NO:2, SEQ ID NO:3, SEQ ID NO:4, SEQ ID NO:5, SEQ ID NO:6, SEQ ID NO:7, or SEQ ID NO:8 using computer programs such as Assay Design Software 1.0.6 (Biotage, Uppsala, Sweden) and Beacon Designer 4.02 (Build 402003) (PREMIER Biosoft International, Palo Alto, Calif.).

PCRs were conducted using the Rotor-Gene 3000 platform (Corbett Research, Sydney, Australia). Parameters for each PCR were as follows. First, an incubation was conducted at 50° C. for 10 minutes for cDNA synthesis. Second, an incubation was performed at 94° C. for 3 minutes to initially denature the DNA, inactivate the iScript reverse transcriptase, and activate the iTaq DNA polymerase. Next, 40 cycles of denaturation (94° C. for 15 seconds), and annealing and extension (47° C. for 45 seconds) were performed with fluorescence acquisition (excitation at 470 nM and emission at 510 nM) immediately following the first cycle. Fluorescence curves were analyzed with dynamic-tube normalization, slope correction, and automatic threshold determination by a best-fit line of three concentrations of a positive-control standard RNA using Rotor-Gene version 5.0 software (Corbett Research, Sydney, Australia). The three concentrations of the positive-control standard RNA per reaction were 1×10³, 1×10⁵, and 1×10⁷ copies of the RNA transcript obtained as described in Example 2.

### EXAMPLE 2

### Positive-Control Standard RNA for the PCR

A plasmid used to generate positive-control standard RNA by in-vitro transcription was obtained by the PCR amplification of 200 ng of RNA extracted from nasal aspirates containing human metapneumovirus isolate 27-2A (University of Iowa, Iowa City, Iowa). Parameters for this PCR were as follows: an initial incubation at 50° C. for 10 minutes; followed by an incubation at 94° C. for 3 minutes; followed by 40 cycles of incubation at 94° C. for 1 minute, 52° C. for 1 minute, and 72° C. for 2 minutes; followed by a final incubation at 72° C. for 10 minutes. All other conditions for this reaction were as described in Example 1. The resulting amplicon was cloned into the pCR®2.1-TOPO® vector (Invitrogen, Carlsbad, Calif.) to produce pMPVFgene2ALG. Positive-control standard RNA was generated by run-off transcription of linearized pMPVFgene2ALG as the template using the Ribomax™ In-Vitro Transcription System (Promega, Madison, Wis.).

### EXAMPLE 3

### Precision of the PCR

Each of five technicians independently assessed the precision of the PCR utilizing the combination of Primer MPV6-Forward, Primer MPV6-Reverse, and Probe MPV6-1 by attempting to conduct real-time PCRs using RNA obtained from clinical specimens (i.e., pediatric nasal washes in PBS). Specifically, each technician tested nine reaction mixtures, each containing RNA purified from a clinical specimen known to contain human metapneumovirus, and one reaction mixture containing RNA purified from a clinical specimen known to be free of human metapneumovirus. As summarized in Table 1, all five technicians correctly determined which of the ten reaction mixtures contained RNA from human metapneumovirus and which did not, even though they did not know the identity of the template RNA in each reaction mixture before attempting to conduct the PCRs.

TABLE 1

| | | Technician | | | | |
|---|---|---|---|---|---|---|
| Sample | Expected | A | B | C | D | E |
| 1 | +* | + | + | + | + | + |
| 2 | + | + | + | + | + | + |
| 3 | + | + | + | + | + | + |
| 4 | + | + | + | + | + | + |
| 5 | + | + | + | + | + | + |
| 6 | + | + | + | + | + | + |
| 7 | + | + | + | + | + | + |
| 8 | + | + | + | + | + | + |
| 9 | + | + | + | + | + | + |
| 10 | −** | − | − | − | − | − |
| Positive Control | + | + | + | + | + | + |
| (1 × 10³ copies of RNA | | | | | | |
| transcript)*** | | | | | | |
| Positive Control | + | + | + | + | + | + |
| (1 × 10⁵ copies of RNA | | | | | | |
| transcript)*** | | | | | | |
| Positive Control | + | + | + | + | + | + |
| (1 × 10⁷ copies of RNA | | | | | | |
| transcript)*** | | | | | | |
| Negative Control | − | − | − | − | − | − |
| (no template DNA) | | | | | | |

*A “+” indicates the presence of RNA of human metapneumovirus in the sample.
**A “−” indicates the absence of RNA of human metapneumovirus in the sample.
***RNA transcript was generated using pMPVFgene2ALG as a template as described in Example 2.

### EXAMPLE 4

### Specificity of the PCR

The precision of the PCR utilizing the combination of Primer MPV6-Forward, Primer MPV6-Reverse, and Probe MPV6-1 was assessed by attempting to conduct real-time PCRs using RNA obtained from each of the pathogens listed in Table 2. Each of these pathogens was purchased from ATCC®. Parameters for each PCR were as follows: an initial incubation at 50° C. for 10 minutes; followed by an incubation at 94° C. for 3 minutes; followed by 40 cycles of incubation at 94° C. for 15 seconds, and 47° C. for 45 seconds. All other conditions for these reactions were as described in Example 1. As indicated in Table 2, no PCR amplification was observed in either of two reaction mixtures containing RNA from each of the pathogens.

TABLE 2

| | ATCC ® | | |
|---|---|---|---|
| Sample (in duplicate) | Number | Expected | Observed |
| Actinomyces israelii | 12597 | −* | − |
| Adenovirus 10 | VR-1087 | − | − |
| Aspergillus fumigatus | 14110 | − | − |
| Atopobium vaginae | BAA-55 | − | − |
| Babesia microti | 30222 | − | − |
| Bacteroides fragilis | 23745 | − | − |
| Bartonella bacilliformis | 35686 | − | − |
| Bartonella henselae | 49882 | − | − |
| BK polyomavirus | VR-837 | − | − |
| Bordetella pertussis | 15989 | − | − |
| Borellia burgdorferi | 35210 | − | − |
| Brucella ovis | 25840 | − | − |
| Candida albicans | 11651 | − | − |
| Candida glabrata | 2001 | − | − |
| Candida krusei | 14243 | − | − |
| Candida lusitaniae | 24350 | − | − |
| Candida parapsilosis | 22019 | − | − |
| Candida tropicalis | 13803 | − | − |
| Candida utilis | 9226 | − | − |
| Canine herpesvirus | VR-552 | − | − |
| Chlamydophila pneumoniae | VR-1356 | − | − |
| Chlamydia trachomatis | VR-901B | − | − |
| Cytomegalovirus | VR-807 | − | − |
| Epstein-Barr virus | CRL-5950 | − | − |
| Escherichia coli | 700336 | − | − |
| Haemophilus ducreyi | 51622 | − | − |
| Haemophilus parainfluenza | 7901 | − | − |
| Heliobacter pylori | 43579 | − | − |
| Herpes simplex virus 1 | VR-1544 | − | − |
| Herpes simplex virus 2 | VR-734 | − | − |
| Human herpesvirus 6B | VR-1467 | − | − |
| Human rhinovirus 6 | VR-1116PI/GP | − | − |
| Human rhinovirus 11 | VR-1121 | − | − |
| Human papillomavirus 16 | CRL-1550 | − | − |
| Influenza B virus | VR-101 | − | − |
| Legionella pneumophila | 33152 | − | − |
| Lymphogranuloma | VR-903 | − | − |
| venereum type III | | | |
| Mobiluncus curtisii | 35241 | − | − |
| Mobiluncus mulieris | 35243 | − | − |
| Mycoplasma genitalium | 33530 | − | − |
| Mycoplasma hominis | 14027 | − | − |
| Mycoplasma pneumoniae | 15377 | − | − |
| Parainfluenza 3 | VR-93 | − | − |
| Streptococcus agalactiae | BAA-611 | − | − |
| Trichosporan cutaneum | 4151 | − | − |
| Trichomonas vaginalis | 30246 | − | − |
| Ureaplasma urealyticum | 27618 | − | − |

| | | |
|---|---|---|
| Positive Control | +*** | + |
| (1 × 10³ copies of RNA transcript)** | | |
| Positive Control | + | + |
| (1 × 10⁵ copies of RNA transcript)** | | |
| Positive Control | + | + |
| (1 × 10⁷ copies of RNA transcript)** | | |
| Negative Control | − | − |
| (no template DNA) | | |

*A “−” indicates the absence of RNA of human metapneumovirus in the sample.
**RNA transcript was generated using pMPVFgene2ALG as a template as described in Example 2.
***A “+” indicates the presence of RNA of human metapneumovirus in the sample.

1. An isolated oligonucleotide comprising 8 nucleotides and being capable of hybridizing under highly stringent hybridization conditions to at least part of a segment of a polynucleotide, wherein the segment consists of nucleotides 661 through 683 of SEQ ID NO:2, SEQ ID NO:4, SEQ ID NO:6, or SEQ ID NO:8, nucleotides 623 through 651 of SEQ ID NO:2, SEQ ID NO:4, SEQ ID NO:6, or SEQ ID NO:8, nucleotides 1078 through 1098 of SEQ ID NO:1, SEQ ID NO:3, SEQ ID NO:5, or SEQ ID NO:7, or nucleotides 970 through 998 of SEQ ID NO:1, SEQ ID NO:3, SEQ ID NO:5, or SEQ ID NO:7.

2. The oligonucleotide of claim 1 comprising at least 10 nucleotides.

3. The oligonucleotide of claim 2 comprising at least 12 nucleotides.

4. The oligonucleotide of claim 3 comprising at least 14 nucleotides.

5. The oligonucleotide of claim 4 comprising at least 16 nucleotides.

6. The oligonucleotide of claim 5 comprising at least 18 nucleotides.

7. The oligonucleotide of claim 1 being from 8 to 50 nucleotides long.

8. The oligonucleotide of claim 7 being from 12 to 24 nucleotides long.

9. The oligonucleotide of claim 7 being from 15 to 50 nucleotides long.

10. The oligonucleotide of claim 9 being from 25 to 35 nucleotides long.

11. The oligonucleotide of claim 1 comprising the nucleotide sequence of SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, or SEQ ID NO:18.

12. The oligonucleotide of claim 11 consisting of the nucleotide sequence of SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, or SEQ ID NO:18.

13. An isolated oligonucleotide comprising 8 nucleotides, wherein, based on the Clustal V or W alignment method using the default parameters, the oligonucleotide is at least 50% identical to the reverse complement of a segment of a polynucleotide, wherein the segment consists of nucleotides 661 through 683 of SEQ ID NO:2, SEQ ID NO:4, SEQ ID NO:6, or SEQ ID NO:8, nucleotides 623 through 651 of SEQ ID NO:2, SEQ ID NO:4, SEQ ID NO:6, or SEQID NO:8, nucleotides 1078 through 1098 of SEQ ID NO:1, SEQ ID NO:3, SEQ ID NO:5, or SEQ ID NO:7, or nucleotides 970 through 998 of SEQ ID NO:1, SEQ ID NO:3, SEQ ID NO:5, or SEQ ID NO:7.

14. The oligonucleotide of claim 13 containing the same number of nucleotides as the segment of the polynucleotide.

15. A composition comprising first and second isolated oligonucleotides, wherein the first oligonucleotide comprises 8 nucleotides and is capable of hybridizing under highly stringent hybridization conditions to a polynucleotide consisting of the nucleotide sequence of SEQ ID NO:2, SEQ ID NO:4, SEQ ID NO:6, or SEQ ID NO:8, and wherein the second oligonucleotide comprises 8 nucleotides and is capable of hybridizing under highly stringent hybridization conditions to a polynucleotide consisting of the nucleotide sequence of SEQ ID NO:1, SEQ ID NO:3, SEQ ID NO:5, or SEQ ID. NO:7.

16. The composition of claim 15 fuirther comprising a third isolated oligonticleotide, wherein the third oligonucleotide comprises 8 nucleotides and is capable of hybridizing under highly stringent hybridization conditions to a polynucleotide consisting of the nucleotide sequence of SEQ ID NO:1, SEQ ID NO:2, SEQ ID NO:3, SEQ ID NO:4, SEQ ID NO:5, SEQ ID NO:6, SEQ ID NO:7, or SEQ ID NO:8.

17. A composition comprising first and second isolated oligonucleotides,
wherein the first oligonucleotide comprises 8 nucleotides and is at least 50% identical to a segment of a first polynucleotide based on the Clustal V or W alignment method using the default parameters,
wherein the first polynucleotide consists of the nucleotide sequence of SEQ ID NO:1, SEQ ID NO:3, SEQ ID NO:5, or SEQ ID NO:7,
wherein the second oligonucleotide comprises 8 nucleotides and is at least 50% identical to a segment of a second polynucleotide based on the Clustal V or W alignment method using the default parameters, and
wherein the second polynucleotide consists of the nucleotide sequence of SEQ ID NO:2, SEQ ID NO:4, SEQ ID NO:6, or SEQ ID NO:8.

18. The composition of claim 17 further comprising a third isolated oligonucleotide, wherein the third oligonucleotide comprises 8 nucleotides and is at least 50% identical to a segment of a polynucleotide based on the Clustal V or W alignment method using the default parameters, wherein the polynucleotide consists of the nucleotide sequence of SEQ ID NO:1, SEQ ID NO:2, SEQ ID NO:3, SEQ ID NO:4, SEQ ID NO:5, SEQ ID NO:6, SEQ ID NO:7, or SEQ ID NO:8.

19. A method for determining whether a sample contains a human metapneumovirus or has an increased likelihood of containing a human metapneumovirus comprising:
(a) providing a vessel containing a composition,
wherein the composition contains first and second primers, and a reverse transcript of an RNA from the sample,
wherein the composition is capable of amplifying, by a polymerase chain reaction, a segment of the reverse transcript to produce an amplicon,
wherein production of the amplicon is primed by the first and second primers,
wherein the first primer is capable of hybridizing under highly stringent hybridization conditions to a polynucleotide consisting of the nucleotide sequence of SEQ ID NO:2, SEQ ID NO:4, SEQ ID NO:6, or SEQ ID NO:8, and
wherein the second primer is capable of hybridizing under highly stringent hybridization conditions to a polynucleotide consisting of the nucleotide sequence of SEQ ID NO:1, SEQ ID NO:3, SEQ ID NO:5, or SEQ ID NO:7,
(b) incubating the vessel under conditions allowing production of the amplicon if the sample contains a human metapneumovirus, and
(c) determining that the sample contains a human metapneumovirus if the amplicon is detected or that the sample has an increased likelihood of containing a human metapneumovirus if the amplicon is detected, or determining that the sample does not contain a human metapneumovirus if the amplicon is not detected or that the sample does not have an increased likelihood of containing a human metapneumovirus if the amplicon is not detected.

20. The method of claim 19, wherein, in (b), the vessel contains an oligonucleotide probe capable of detecting the amplicon if the amplicon is produced in (b).


SEQUENCE LISTING (18 sequences)

SEQ ID NO: 1 - DNA, 1620 bp, human metapneumovirus isolate 00-1
atgtcttggaaagtggtgatcattttttcattgttaataacacctcaacacggtcttaaagagagctacttagaagagtcatgtagcactataactgaaggatatctcagtgttctgaggacaggttggtacaccaatgtttttacactggaggtaggcgatgtagagaaccttacatgtgccgatggacccagcttaataaaaacagaattagacctgaccaaaagtgcactaagagagctcagaacagtttctgctgatcaactggcaagagaggagcaaattgaaaatcccagacaatctagattcgttctaggagcaatagcactcggtgttgcaactgcagctgcagttacagcaggtgttgcaattgccaaaaccatccggcttgaaagtgaagtaacagcaattaagaatgccctcaaaaagaccaatgaagcagtatctacattggggaatggagttcgtgtgttggcaactgcagtgagagagctgaaagattttgtgagcaagaatctaacacgtgcaatcaacaaaaacaagtgcgacattgctgacctgaaaatggccgttagcttcagtcaattcaacagaaggttcctaaatgttgtgcggcaattttcagacaacgctggaataacaccagcaatatctttggacttaatgacagatgctgaactagccagagctgtttccaacatgccaacatctgcaggacaaataaaactgatgttggagaaccgtgcaatggtaagaagaaaagggttcggattcctgataggagtttacggaagctccgtaatttacatggtgcaactgccaatctttggggttatagacacgccttgctggatagtaaaagcagccccttcttgttcaggaaaaaagggaaactatgcttgcctcttaagagaagaccaaggatggtattgtcaaaatgcagggtcaactgtttactacccaaatgaaaaagactgtgaaacaagaggagaccatgtcttttgcgacacagcagcaggaatcaatgttgctgagcagtcaaaggagtgcaacataaacatatctactactaattacccatgcaaagttagcacaggaagacatcctatcagtatggttgcactatctcctcttggggctttggttgcttgctacaagggagtgagctgttccattggcagcaacagagtagggatcatcaagcaactgaacaaaggctgctcttatataaccaaccaagacgcagacacagtgacaatagacaacactgtataccagctaagcaaagttgaaggcgaacagcatgttataaaaggaaggccagtgtcaagcagctttgacccagtcaagtttcctgaagatcaattcaatgttgcacttgaccaagttttcgagagcattgagaacagtcaggccttggtggatcaatcaaacagaatcctaagcagtgcagagaaaggaaacactggcttcatcattgtaataattctaattgctgtccttggctctaccatgatcctagtgagtgtttttatcataataaagaaaacaaagaaacccacaggagcacctccagagctgagtggtgtcacaaacaatggcttcataccacataattag

SEQ ID NO: 2 - DNA, 1620 bp, human metapneumovirus isolate 00-1
ctaattatgtggtatgaagccattgtttgtgacaccactcagctctggaggtgctcctgtgggtttctttgttttctttattatgataaaaacactcactaggatcatggtagagccaaggacagcaattagaattattacaatgatgaagccagtgtttcctttctctgcactgcttaggattctgtttgattgatccaccaaggcctgactgttctcaatgctctcgaaaacttggtcaagtgcaacattgaattgatcttcaggaaacttgactgggtcaaagctgcttgacactggccttccttttataacatgctgttcgccttcaactttgcttagctggtatacagtgttgtctattgtcactgtgtctgcgtcttggttggttatataagagcagcctttgttcagttgcttgatgatccctactctgttgctgccaatggaacagctcactcccttgtagcaagcaaccaaagccccaagaggagatagtgcaaccatactgataggatgtcttcctgtgctaactttgcatgggtaattagtagtagatatgtttatgttgcactcctttgactgctcagcaacattgattcctgctgctgtgtcgcaaaagacatggtctcctcttgtttcacagtctttttcatttgggtagtaaacagttgaccctgcattttgacaataccatccttggtcttctcttaagaggcaagcatagtttcccttttttcctgaacaagaaggggctgcttttactatccagcaaggcgtgtctataaccccaaagattggcagttgcaccatgtaaattacggagcttccgtaaactcctatcaggaatccgaacccttttcttcttaccattgcacggttctccaacatcagttttatttgtcctgcagatgttggcatgttggaaacagctctggctagttcagcatctgtcattaagtccaaagatattgctggtgttattccagcgttgtctgaaaattgccgcacaacatttaggaaccttctgttgaattgactgaagctaacggccattttcaggtcagcaatgtcgcacttgtttttgttgattgcacgtgttagattcttgctcacaaaatctttcagctctctcactgcagttgccaacacacgaactccattccccaatgtagatactgcttcattggtctttttgagggcattcttaattgctgttacttcactttcaagccggatggttttggcaattgcaacacctgctgtaactgcagctgcagttgcaacaccgagtgctattgctcctagaacgaatctagattgtctgggattttcaatttgctcctctcttgccagttgatcagcagaaactgttctgagctctcttagtgcacttttggtcaggtctaattctgtttttattaagctgggtccatcggcacatgtaaggttctctacatcgcctacctccagtgtaaaaacattggtgtaccaacctgtcctcagaacactgagatatccttcagttatagtgctacatgactcttctaagtagctctctttaagaccgtgttgaggtgttattaacaatgaaaaaatgatcaccactttccaagacat

SEQ ID NO: 3 - DNA, 1620 bp, human metapneumovirus isolate CAN97-83
atgtcttggaaagtggtgatcattttttcattgctaataacacctcaacacggtcttaaagagagctacctagaagaatcatgtagcactataactgagggatatcttagtgttctgaggacaggttggtataccaacgtttttacattagaggtgggtgatgtagaaaaccttacatgttctgatggacctagcctaataaaaacagaattagatctgaccaaaagtgcactaagagagctcaaaacagtctctgctgaccaattggcaagagaggaacaaattgagaatcccagacaatctaggtttgttctaggagcaatagcactcggtgttgcaacagcagctgcagtcacagcaggtgttgcaattgccaaaaccatccggcttgagagtgaagtcacagcaattaagaatgccctcaaaacgaccaatgaagcagtatctacattggggaatggagttcgagtgttggcaactgcagtgagagagctgaaagactttgtgagcaagaatttaactcgtgcaatcaacaaaaacaagtgcgacattgatgacctaaaaatggccgttagcttcagtcaattcaacagaaggtttctaaatgttgtgcggcaattttcagacaatgctggaataacaccagcaatatctttggacttaatgacagatgctgaactagccagggccgtttctaacatgccgacatctgcaggacaaataaaattgatgttggagaaccgtgcgatggtgcgaagaaaggggttcggaatcctgataggggtctacgggagctccgtaatttacatggtgcagctgccaatctttggcgttatagacacgccttgctggatagtaaaagcagccccttcttgttccggaaaaaagggaaactatgcttgcctcttaagagaagaccaagggtggtattgtcagaatgcagggtcaactgtttactacccaaatgagaaagactgtgaaacaagaggagaccatgtcttttgcgacacagcagcgggaattaatgttgctgagcaatcaaaggagtgcaacatcaacatatccactacaaattacccatgcaaagtcagcacaggaagacatcctatcagtatggttgcactgtctcctcttggggctctggttgcttgctacaaaggagtaagctgttccattggcagcaacagagtagggatcatcaagcagctgaacaagggttgctcctatataaccaaccaagatgcagacacagtgacaatagacaacactgtatatcagctaagcaaagttgagggtgaacagcatgttataaaaggcagaccagtgtcaagcagctttgatccaatcaagtttcctgaagatcaattcaatgttgcacttgaccaagtttttgagaacattgaaaacagccaggccttggtagatcaatcaaacagaatcctaagcagtgcagagaaagggaatactggcttcatcattgtaataattctaattgctgtccttggctctagcatgatcctagtgagcatcttcattataatcaagaaaacaaagaaaccaacgggagcacctccagagctgagtggtgtcacaaacaatggcttcataccacacagttag

SEQ ID NO: 4 - DNA, 1620 bp, human metapneumovirus isolate CAN97-83
ctaactgtgtggtatgaagccattgtttgtgacaccactcagctctggaggtgctcccgttggtttctttgttttcttgattataatgaagatgctcactaggatcatgctagagccaaggacagcaattagaattattacaatgatgaagccagtattccctttctctgcactgcttaggattctgtttgattgatctaccaaggcctggctgttttcaatgttctcaaaaacttggtcaagtgcaacattgaattgatcttcaggaaacttgattggatcaaagctgcttgacactggtctgccttttataacatgctgttcaccctcaactttgcttagctgatatacagtgttgtctattgtcactgtgtctgcatcttggttggttatataggagcaacccttgttcagctgcttgatgatccctactctgttgctgccaatggaacagcttactcctttgtagcaagcaaccagagccccaagaggagacagtgcaaccatactgataggatgtcttcctgtgctgactttgcatgggtaatttgtagtggatatgttgatgttgcactcctttgattgctcagcaacattaattcccgctgctgtgtcgcaaaagacatggtctcctcttgtttcacagtctttctcatttgggtagtaaacagttgaccctgcattctgacaataccacccttggtcttctcttaagaggcaagcatagtttcccttttttccggaacaagaaggggctgcttttactatccagcaaggcgtgtctataacgccaaagattggcagctgcaccatgtaaattacggagctcccgtagacccctatcaggattccgaacccctttcttcgcaccatcgcacggttctccaacatcaattttatttgtcctgcagatgtcggcatgttagaaacggccctggctagttcagcatctgtcattaagtccaaagatattgctggtgttattccagcattgtctgaaaattgccgcacaacatttagaaaccttctgttgaattgactgaagctaacggccatttttaggtcatcaatgtcgcacttgtttttgttgattgcacgagttaaattcttgctcacaaagtctttcagctctctcactgcagttgccaacactcgaactccattccccaatgtagatactgcttcattggtcgttttgagggcattcttaattgctgtgacttcactctcaagccggatggttttggcaattgcaacacctgctgtgactgcagctgctgttgcaacaccgagtgctattgctcctagaacaaacctagattgtctgggattctcaatttgttcctctcttgccaattggtcagcagagactgttttgagctctcttagtgcacttttggtcagatctaattctgtttttattaggctaggtccatcagaacatgtaaggttttctacatcacccacctctaatgtaaaaacgttggtataccaacctgtcctcagaacactaagatatccctcagttatagtgctacatgattcttctaggtagctctctttaagaccgtgttgaggtgttattagcaatgaaaaaatgatcaccactttccaagacat

SEQ ID NO: 5 - DNA, 1620 bp, human metapneumovirus isolate NL/1/99
atgtcttggaaagtgatgatcatcatttcgttactcataacaccccagcacgggctaaaggagagttatttggaagaatcatgtagtactataactgagggatacctcagtgttttaagaacaggctggtacactaatgtcttcacattagaagttggtgatgttgaaaatcttacatgtactgatggacctagcttaatcaaaacagaacttgatctaacaaaaagtgctttaagggaactcaaaacagtctctgctgatcagttggcgagagaggagcaaattgaaaatcccagacaatcaagatttgtcttaggtgcgatagctctcggagttgctacagcagcagcagtcacagcaggcattgcaatagccaaaaccataaggcttgagagtgaggtgaatgcaattaaaggtgctctcaaacaaactaatgaagcagtatccacattagggaatggtgtgcgggtcctagccactgcagtgagagagctaaaagaatttgtgagcaaaaacctgactagtgcaatcaacaggaacaaatgtgacattgctgatctgaagatggctgtcagcttcagtcaattcaacagaagatttctaaatgttgtgcggcagttttcagacaatgcagggataacaccagcaatatcattggacctgatgactgatgctgagttggccagagctgtatcatacatgccaacatctgcagggcagataaaactgatgttggagaaccgcgcaatggtaaggagaaaaggatttggaatcctgataggggtctacggaagctctgtgatttacatggttcaattgccgatctttggtgtcatagatacaccttgttggatcatcaaggcagctccctcttgctcagaaaaaaacgggaattatgcttgcctcctaagagaggatcaagggtggtattgtaaaaatgcaggatctactgtttactacccaaatgaaaaagactgcgaaacaagaggtgatcatgttttttgtgacacagcagcagggatcaatgttgctgagcaatcaagagaatgcaacatcaacatatctactaccaactacccatgcaaagtcagcacaggaagacaccctataagcatggttgcactatcacctctcggtgctttggtggcttgctataaaggggtaagctgctcgattggcagcaattgggttggaatcatcaaacaattacccaaaggctgctcatacataaccaaccaggatgcagacactgtaacaattgacaataccgtgtatcaactaagcaaagttgaaggtgaacagcatgtaataaaagggagaccagtttcaagcagttttgatccaatcaagtttcctgaggatcagttcaatgttgcgcttgatcaagtcttcgaaagcattgagaacagtcaggcactagtggaccagtcaaacaaaattctaaacagtgcagaaaaaggaaacactggtttcattatcgtagtaattttggttgctgttcttggtctaaccatgatttcagtgagcatcatcatcataatcaagaaaacaaggaagcccacaggagcacctccagagctgaatggtgtcaccaacggcggtttcataccacatagttag

SEQ ID NO: 6 - DNA, 1200 bp, human metapneumovirus isolate NL/1/99
ctaactatgtggtatgaaaccgccgttggtgacaccattcagctctggaggtgctcctgtgggcttccttgttttcttgattatgatgatgatgctcactgaaatcatggttagaccaagaacagcaaccaaaattactacgataatgaaaccagtgtttcctttttctgcactgtttagaattttgtttgactggtccactagtgcctgactgttctcaatgctttcgaagacttgatcaagcgcaacattgaactgatcctcaggaaacttgattggatcaaaactgcttgaaactggtctcccttttattacatgctgttcaccttcaactttgcttagttgatacacggtattgtcaattgttacagtgtctgcatcctggttggttatgtatgagcagcctttgggtaattgtttgatgattccaacccaattgctgccaatcgagcagcttacccctttatagcaagccaccaaagcaccgagaggtgatagtgcaaccatgcttatagggtgtcttcctgtgctgactttgcatgggtagttggtagtagatatgttgatgttgcattctcttgattgctcagcaacattgatccctgctgctgtgtcacaaaaaacatgatcacctcttgtttcgcagtctttttcatttgggtagtaaacagtagatcctgcatttttacaataccacccttgatcctctcttaggaggcaagcataattcccgtttttttctgagcaagagggagctgccttgatgatccaacaaggtgtatctatgacaccaaagatcggcaattgaaccatgtaaatcacagagcttccgtagacccctatcaggattccaaatccttttctccttaccattgcgcggttctccaacatcagttttatctgccctgcagatgttggcatgtatgatacagctctggccaactcagcatcagtcatcaggtccaatgatattgctggtgttatccctgcattgtctgaaaactgccgcacaacatttagaaatcttctgttgaattgactgaagctgacagccatcttcagatcagcaatgtcacatttgttcctgttgattgcactagtcaggtttttgctcacaaattcttttagctctctcactgcagtggctaggacccgcacaccattccctaatgtggatactgcttcattagtttgtttgag

SEQ ID NO: 7 - DNA, 1620 bp, human metapneumovirus isolate CAN98-75
atgtcttggaaagtgatgattatcatttcgttactcataacacctcagcacggactaaaggaaagttatttagaagaatcatgtagtactataactgaaggatatctcagtgttttaagaacaggttggtacaccaatgtctttacattagaagttggtgatgttgaaaatcttacatgtactgatggacctagcttaatcaaaacagaacttgacctaaccaaaagtgctctaagagaactcaaaacagtttctgctgatcagttagcgagagaagaacaaattgaaaatcccagacaatcaaggtttgtcctaggtgcaatagctcttggtgttgccacagcagcagcagtcacagcaggcattgcgatagccaaaaccataaggcttgagagtgaagtgaatgcaatcaaaggtgctctcaaaacaaccaatgaggcagtatccacactaggaaatggagtgcgagtcctagccaccgcagtaagagagctgaaagaatttgtgagcaaaaacctgactagtgcaattaacaagaacaaatgtgacattgctgatctgaagatggctgtcagcttcagtcaattcaacagaagattcctaaatgttgtgcggcagttttcagacaatgcagggataacaccagcaatatcattggacctaatgactgatgctgagctggccagagctgtatcatacatgccaacatctgcaggacagataaaactaatgttagagaaccgtgcaatggtgaggagaaaaggatttggaatcttgataggggtctacggaagctccgtgatttacatggtccagctgccgatctttggtgtcatagatacaccttgttggataatcaaggcagctccctcttgttcagaaaaagatggaaactatgcttgcctcctaagagaggatcaagggtggtattgtaaaaatgcaggatccactgtttactacccaaataaaaaagactgcgaaacaagaggtgatcatgttttttgtgacacagctgcagggatcaatgttgctgagcaatcaagagaatgcaacatcaacatatctacaaccaactacccatgcaaagtcagcacaggaagacaccctatcagcatggttgcactatcacctctcggtgctttggtggcttgctacaaaggggttagctgttcaattggcagtaatcgggttggaataatcaaacaactacctaaaggctgctcatacataactaaccaggacgcagacactgtaacaattgacaacactgtgtatcaactaagcaaagttgagggtgaacagcatgtaataaaagggagaccagtttcaagcagtttcgatccaatcaagtttcctgaggatcagttcaatgttgcgcttgatcaagtctttgaaagcattgaaaacagtcaagcactagtggaccagtcaaacaaaattctgaacagtgcagaaaaaggaaacactggcttcattattgtaataattttgattgctgttcttgggttaaccatgatttcagtgagcatcatcatcataatcaaaaaaacaaggaaacccacaggggcacctccagagctgaatggtgttaccaacggcggttttataccgcatagttag

SEQ ID NO: 8 - DNA, 1620 bp, human metapneumovirus isolate CAN98-75
ctaactatgcggtataaaaccgccgttggtaacaccattcagctctggaggtgcccctgtgggtttccttgtttttttgattatgatgatgatgctcactgaaatcatggttaacccaagaacagcaatcaaaattattacaataatgaagccagtgtttcctttttctgcactgttcagaattttgtttgactggtccactagtgcttgactgttttcaatgctttcaaagacttgatcaagcgcaacattgaactgatcctcaggaaacttgattggatcgaaactgcttgaaactggtctcccttttattacatgctgttcaccctcaactttgcttagttgatacacagtgttgtcaattgttacagtgtctgcgtcctggttagttatgtatgagcagcctttaggtagttgtttgattattccaacccgattactgccaattgaacagctaacccctttgtagcaagccaccaaagcaccgagaggtgatagtgcaaccatgctgatagggtgtcttcctgtgctgactttgcatgggtagttggttgtagatatgttgatgttgcattctcttgattgctcagcaacattgatccctgcagctgtgtcacaaaaaacatgatcacctcttgtttcgcagtcttttttatttgggtagtaaacagtggatcctgcatttttacaataccacccttgatcctctcttaggaggcaagcatagtttccatctttttctgaacaagagggagctgccttgattatccaacaaggtgtatctatgacaccaaagatcggcagctggaccatgtaaatcacggagcttccgtagacccctatcaagattccaaatccttttctcctcaccattgcacggttctctaacattagttttatctgtcctgcagatgttggcatgtatgatacagctctggccagctcagcatcagtcattaggtccaatgatattgctggtgttatccctgcattgtctgaaaactgccgcacaacatttaggaatcttctgttgaattgactgaagctgacagccatcttcagatcagcaatgtcacatttgttcttgttaattgcactagtcaggtttttgctcacaaattctttcagctctcttactgcggtggctaggactcgcactccatttcctagtgtggatactgcctcattggttgttttgagagcacctttgattgcattcacttcactctcaagccttatggttttggctatcgcaatgcctgctgtgactgctgctgctgtggcaacaccaagagctattgcacctaggacaaaccttgattgtctgggattttcaatttgttcttctctcgctaactgatcagcagaaactgttttgagttctcttagagcacttttggttaggtcaagttctgttttgattaagctaggtccatcagtacatgtaagattttcaacatcaccaacttctaatgtaaagacattggtgtaccaacctgttcttaaaacactgagatatccttcagttatagtactacatgattcttctaaataactttcctttagtccgtgctgaggtgttatgagtaacgaaatgataatcatcactttccaagacat

SEQ ID NO: 9 - DNA, 20 bp, human metapneumovirus isolate 00-1
atgcagggtcaactgtttac

SEQ ID NO: 10 - DNA, 20 bp, human metapneumovirus isolate NL/1/99
atgcaggatctactgtttac

SEQ ID NO: 11 - DNA, 20 bp, human metapneumovirus CAN98-75
atgcaggatccactgtttac

SEQ ID NO: 12 - DNA, 20 bp, human metapneumovirus
atgcaggrtchactgtttac

SEQ ID NO: 13 - DNA, 21 bp, human metapneumovirus 00-1
tcctgtgctaactttgcatgg

SEQ ID NO: 14 - DNA, 21 bp, human metapneumovirus CAN97-83
tcctgtgctgactttgcatgg

SEQ ID NO: 15 - DNA, 21 bp, human metapneumovirus
tcctgtgctractttgcatgg

SEQ ID NO: 16 - DNA, 29 bp, human metapneumovirus isolate 00-1
aaagactgtgaaacaagaggagaccatgt

SEQ ID NO: 17 - DNA, 29 bp, human metapneumovirus isolate NL/1/99
aaagactgcgaaacaagaggtgatcatgt

SEQ ID NO: 18 - DNA, 29 bp, human metapneumovirus
aaagactgygaaacaagaggwgaycatgt